(19) United States
(12) Patent Application Publication
(10) Pub. No.: US 2015/0022362 A1
(43) Pub. Date: Jan. 22, 2015

(54) WEARABLE DEVICE CONFIGURED TO DETECT IMPROPER MOVEMENT OF A KNEE AND ALERT WEARERS TO THE IMPROPER MOVEMENT

(71) Applicant: Smart Sport Systems, LLC, Roanoke, VA (US)

(72) Inventors: Mark Stephen LUCAS, Roanoke, VA (US); Wendy Tignor LUCAS, Roanoke, VA (US); Louis CIRILLO, Blacksburg, VA (US); Calleigh A. LUCAS, Roanoke, VA (US); Claiborne J. LUCAS, Roanoke, VA (US)

(73) Assignee: Smart Sport Systems, LLC, Roanoke, VA (US)

(21) Appl. No.: 13/948,089

(22) Filed: Jul. 22, 2013

(51) Int. Cl. A61B 5/11

(52) U.S. Cl. CPC A61B 5/1116
USPC 340/573.7

(57) ABSTRACT

A wearable device may be configured to detect and alert wearers to improper movements at a body joint based on a comparison of a lateral angle and a bend angle of the body joint and/or based on measurements customized for an individual wearer of the wearable device. For example, a wearable device may determine a bend angle and a corresponding lateral angle of the knee. The wearable device may compare the determined bend angle and lateral angle with stored thresholds customized for the wearer. The wearable device may select a threshold from the stored thresholds with a bend angle that matches the determined bend angle. The wearable device may then determine whether the determined lateral angle matches and/or exceeds one or more lateral angles associated with the bend angle of the threshold. The wearable device may provide feedback responsive to a determination that the threshold was exceeded.

### FIELD OF THE INVENTION

The invention relates to a wearable device configured to detect and alert wearers to improper movements at a body joint based on a comparison of a lateral angle and a bend angle of the body joint and/or based on measurements customized for an individual wearer of the wearable device.

### BACKGROUND OF THE INVENTION

Generally speaking, braces have been used to provide support to a body joint, such as a knee, ankle, wrist, or elbow. A body joint comprises a physical connection between two or more bones in a human body. A body joint may be classified as a fibrous (immovable) joint, a cartilaginous (partially movable) joint, or a synovial (freely movable) joint. A synovial body joint is typically constructed to allow movement between the connected bones. For example, a knee joint may comprise a physical connection between the femur (e.g., first bone), the tibia (e.g., second bone), and patella (e.g., third bone) that allows movement of the connected bones.

Body joints may also be classified as simple joints or compound joints. A simple joint may comprise a body joint that connects two bones. Examples of simple synovial joints include the hip, the shoulder, and/or other joints at which two bones connect. A compound body joint may comprise a body joint that connects more than two bones. Examples of compound synovial body joints include the knee, the wrist, the elbow, and/or other joints at which more than two bones are movably connected. Compound synovial joints generally tend to comprise a more complex structure and have more restricted types of movement and more restricted bounds of movement for a type of movement than simple joints. Accordingly, compound synovial joints are generally more susceptible to injury during movement.

A brace for a body joint may be used to facilitate healing of an injured body joint or to prevent a body joint from engaging in movement that may cause injury. For example, a brace may constrict movement of an injured joint to facilitate a faster healing process. In another example, a brace may be used in sports to provide additional support to a joint when the wearer is engaging in more rigorous use of the joint than in normal activities. In yet another example, a brace may be used to help the wearer engage in a preferred type of movement during sports and/or other physical activity.

Some braces may be used in conjunction with a sensor to provide certain feedback to a wearer. However, these braces have various drawbacks. For example, at least some of these sensors have generic thresholds (e.g., not user specific) and/or thresholds preset by users, doctors, coaches, and/or other persons interested in a wearer's joint movement. These devices may be restricted to alerting a wearer to movement exceeding these generic or preset thresholds. Further, some of these devices may only sense a single type of movement at the body joint (e.g., a relative motion between two bones connected by the body joint).

Conventional braces suffer from these and other drawbacks.

### SUMMARY OF THE INVENTION

The invention relates to a wearable device configured to detect and alert a wearer of the device to a movement of the knee that approaches or exceeds a threshold that is customized for the wearer. A threshold may comprise a combination of a bend angle and one or more lateral angle values at which the knee makes or is about to make an improper movement. An improper movement comprises a movement that may result in injury, pain, and/or other undesirable consequence. A bend angle of a knee may be an angle formed on the transverse axis by a first bone (e.g., the tibia) relative to a second bone (e.g., the femur). A lateral angle of the knee may be an angle formed on the lateral axis by a horizontal displacement of the first bone (e.g., the tibia) relative to the second bone (e.g., the femur). A transverse axis may be perpendicular to a vertical axis and perpendicular to a horizontal axis. For example, a knee may move along the transverse axis by, for example, flexion and extension. A knee may move along the vertical axis by, for example, elevation and depression. A knee may move along the lateral axis by, for example, abduction and adduction.

For an individual bend angle, there may be more than one corresponding lateral angle at which the knee may make an improper movement. For example, the individual angle may be associated with a corresponding inward lateral angle, at which the knee makes an improper movement. The inward lateral angle may be an angle formed on the lateral axis by a horizontal inward displacement of the first bone (e.g., the tibia) relative to the second bone (e.g., the femur), where the first bone has moved inward (e.g., towards the midline of the body) relative to the second bone. The individual angle may also be associated with a corresponding outward lateral angle, at which the knee makes an improper movement. The outward lateral angle may be an angle formed on the lateral axis by a horizontal outward displacement of the first bone (e.g., the tibia) relative to the second bone (e.g., the femur), where the first bone has moved outwards (e.g., away from the midline of the body) relative to the second bone. Accordingly, a threshold may comprise a bend angle value, an inward lateral angle value, and an outward lateral angle value.

In some examples, a threshold may comprise an individual bend angle associated with multiple inward lateral angles and multiple outward lateral angle values. For example, a combination of the individual bend angle and a first inward lateral angle of the plurality of inward lateral angles may be associated with an improper movement of the knee. A combination of the individual bend angle and one or more other inward lateral angles may be associated with a movement of the knee in a direction in which an improper movement may occur. The threshold may also comprise, for an individual lateral angle, an indication whether the combination of the individual bend angle and the individual lateral angle is associated with an improper movement or with a potential occurrence of an improper movement. In some examples, the threshold may also comprise, for an individual lateral angle associated with the individual bend angle, feedback characteristics associated with feedback to provide to the wearer responsive to a determination that the threshold has been exceeded relative to the individual lateral angle. Feedback characteristics may comprise a number of types of feedback, one or more types of feedback, an intensity of feedback for individual ones of the one or more types of feedback, a length of time for which individual ones of the one or more types of feedback should be provided, and/or other characteristics related to providing feedback to the wearer of the device.

The wearable device may determine a bend angle and a corresponding lateral angle of the knee. For example, the bend angle and corresponding lateral angle of the knee may be determined by sensing the relative position of the tibia and the femur. The wearable device may compare the determined bend angle and lateral angle with stored thresholds customized for the wearer. The thresholds may be stored on a non-transitory electronic storage media coupled to the wearable device, may be stored on a mobile device of the wearer, may be stored at a remote data repository, and/or may be stored at another storage communicably coupled to the wearable device. The wearable device may select a threshold from the plurality of thresholds with a bend angle that matches the determined bend angle. The wearable device may then determine whether the determined lateral angle matches and/or exceeds one or more lateral angles associated with the bend angle of the threshold. The wearable device may provide feedback based on the largest lateral angle associated with the bend angle of the threshold that the determined lateral angle matches and/or exceeds.

As mentioned above, the stored thresholds may be customized for a wearer of the wearable device. The wearable device may be configured to obtain baseline information for the wearer's knee. For example, the wearable device may determine a plurality of bend angles and corresponding lateral angles of the wearer's knees during a movement of the leg from a straightened position to a bent position. The straightened position may comprise a position in which the tibia and the femur form a 180 degree angle relative to each other on the transverse axis. The bent position may comprise a position in which the tibia and the femur achieve a maximum bend along the transverse axis (and in which the bend angle of the knee along the transverse axis would be determined), a position in which the tibia and the femur comprise a 90 degree angle relative to each other on the transverse axis, and/or another position in which the tibia is bent with respective to the femur. In some examples, the baseline information may comprise a bend angle and a lateral angle for each possible bend angle from the straightened position to the bent position. In some examples, the baseline information may comprise a bend angle and a lateral angle for a plurality of possible bend angles from the straightened position to the bent position.

The baseline information may be used to determine the stored threshold values. A threshold may be determined for an individual bend angle based on the value of the individual bend angle and the value of the corresponding lateral angle. The value of the corresponding lateral angle may be used, for example, as a median value to determine an inward lateral angle at which the knee would be engaging in improper movement and an outward lateral angle at which the knee would be engaging in improper movement. In some examples, a plurality of inward lateral angles and a plurality of outward lateral angles may be determined for the threshold as well.

According to an aspect of the invention, a wearable device may comprise, for example, a compliant sleeve, a first sensor, a non-transitory electronic storage media, a user input component, an accelerometer, a physical processor, a feedback device, and/or other components.

The compliant sleeve may comprise a sleeve with an opening at a first end and an opening at a second end opposite to the first end, where the sleeve may be configured to encompass the knee of a wearer of the wearable device. The sleeve may comprise a fabric sleeve. In some implementations, the physical processor may be coupled with the fabric of the sleeve.

The first sensor may be coupled to the sleeve. The first sensor may be configured to sense movement of the knee. In some examples, sensory information from the first sensor may comprise information related to a relative position of the tibia and the femur. In some examples, the sensory information may comprise information related to a first bend angle and a first lateral angle formed by the tibia and the femur at the first time. In some implementations, the sensory information may comprise information related to a first bend angle and a first lateral angle sensed at a first time and a second bend angle and a second lateral angle sensed at a second time after the first time. The sensory information may comprise a bend angle and corresponding lateral angle for each angle sensed during movement of the knee.

The first sensor may be configured to transmit the sensory information related to the movement of the knee. The sensor may transmit the sensory information to the physical processor, to a non-transitory electronic storage media coupled to the wearable device, to a remote data repository, and/or to another storage configured to store the sensory information. In some implementations, multiple sensors may be used.

The accelerometer may be configured to detect acceleration of movement of the knee and store the detected acceleration information. The acceleration information may comprise, for example, an acceleration value associated with the acceleration of the knee at a first time. In some examples, the acceleration information may comprise a first acceleration value associated with acceleration of the knee at a first time and a second acceleration value associated with acceleration of the knee at a second time after the first time. In some implementations, the acceleration information may comprise a respective acceleration value associated with each bend angle and corresponding lateral angle in the sensory information.

The non-transitory electronic storage media may be configured to store thresholds for the wearer of the wearable device. In some implementations, the electronic storage media may be configured to store sensory information obtained via the wearable device.

The user input component may be configured to receive input based on wearer interaction with the user input component. The user input component may generate a signal based on the received input. The user input component may be coupled to an outer surface of the wearable device. The signal generated by the user input component may indicate one or more of: an indication to begin sensing the movement of the body joint, an indication to stop sensing the movement of the body joint, an indication to obtain baseline information from the wearer, and/or another indication to perform functionality of the wearable device.

The physical processor may be coupled to the sleeve and may be communicably coupled to the first sensor and communicably coupled to the electronic storage media. The physical processor may be configured to execute one or more program modules. The program modules may comprise, for example, a sensor reception module, a movement detection module, a signal generation module, a threshold determination module, a customization module, and/or other computer program modules.

The sensor reception module may be configured to receive the sensory information. In some implementations, the sensor reception module may determine a first bend angle and a first lateral angle based on the sensory information. In some implementations, the sensor reception module may determine a plurality of bend angles and corresponding lateral angles based on the sensory information. For example, the sensor reception module may determine a bend angle and corresponding lateral angle for each angle sensed during movement of the knee.

The movement detection module may be configured to determine whether the first bend angle and the first lateral angle exceed a threshold for the wearer of the wearable device. The movement detection module may select a threshold from the plurality of thresholds with a bend angle that matches the first bend angle. The movement detection module may determine whether the first lateral angle is an inward lateral angle or an outward lateral angle. Responsive to a determination that the first lateral angle is an inward lateral angle, the movement detection module may determine whether the first lateral angle matches and/or exceeds one or more inward lateral angles associated with the bend angle of the threshold. Responsive to a determination that the first lateral angle is an outward lateral angle, the movement detection module may determine whether the first lateral angle matches and/or exceeds one or more outward lateral angles associated with the bend angle of the threshold.

In some implementations where the sensory information comprises a plurality of bend angles and corresponding lateral angles sensed at a respective plurality of consecutive times, the movement detection module may be configured to determine, for individual ones of the bend angles and corresponding lateral angles, whether the individual bend angle and corresponding lateral angle exceed a corresponding threshold for the wearer of the wearable device.

In some implementations where the wearable device comprises an accelerometer, the movement detection module may be configured to determine whether the first lateral angle and the first bend angle exceed a corresponding threshold for the wearer of the wearable device based on acceleration information for the knee at the first time.

The signal generation module may be configured to generate a signal indicating that the knee has made an improper movement, responsive to a determination by the movement detection module that the first lateral angle matches and/or exceeds one or more lateral angles associated with the bend angle of the threshold. The signal generation module may generate a signal based on the largest lateral angle associated with the bend angle of the threshold that the first lateral angle matches and/or exceeds. The signal generation module may transmit the generated signal to a feedback device coupled to the wearable device, to cause the feedback device to provide feedback based on the generated signal. In some implementations, based on stored feedback characteristics associated with a largest lateral angle of the threshold which the first lateral angle matched and/or exceeded, the signal generation module may generate one or more signals to cause the feedback device (and/or one or more other feedback devices coupled to and/or communicably coupled to the wearable device) to generate feedback according to the stored feedback characteristics.

The threshold determination module may be configured to revise a threshold based on the bend angle and one or more lateral angles of the threshold and on the stored sensory information. The threshold determination module may determine that a second lateral angle associated with a second bend angle sensed at a second time did not match and/or exceed a corresponding second threshold. The threshold determination module may determine that a third lateral angle associated with a third bend angle sensed at a third time immediately consecutive to the second time matched and/or exceeded a corresponding third threshold. The threshold determination module may revise the threshold for the second bend angle to include the second lateral angle with an indication that the combination of the second bend angle and the second lateral angle is associated with a potential occurrence of an improper movement.

In some implementations where the wearable device comprises the accelerometer, threshold determination module may be configured to revise a threshold based on the bend angle and one or more lateral angles of the threshold, the stored sensory information, and acceleration information corresponding to the movement of the knee at the bend angle. The threshold determination module may determine that a second lateral angle associated with a second bend angle sensed at a second time did not match and/or exceed a corresponding second threshold. The threshold determination module may determine that a third lateral angle associated with a third bend angle sensed at a third time immediately consecutive to the second time matched and/or exceeded a corresponding third threshold. The threshold determination module may further determine, based on the acceleration information associated with the second bend angle and the second lateral angle, that the knee would necessarily engage in movement in which it formed the third bend angle and the third lateral angle responsive to the knee forming the second bend angle and the second lateral angle when the knee moved at an acceleration included in the acceleration information. Responsive to that determination, the threshold determination module may revise the threshold for the second bend angle to include the second lateral angle with an indication that the combination of the second bend angle and the second lateral angle is associated with a potential occurrence of an improper movement.

The customization module may be configured to obtain baseline information from the wearer responsive to a determination that a signal to obtain baseline information from the wearer was generated. The customization module may be configured to determine thresholds for the wearer based on the baseline information. In some implementations, the wearable device may be unable to provide feedback to the wearer based on the movement of the wearer's knee until the wearable device obtains baseline information for the wearer. In some implementations, the electronic storage media of the wearable device may store a plurality of initial thresholds. The wearable device may revise, replace, and/or otherwise change the plurality of initial thresholds based on receiving baseline information (e.g., via the customization module), revising the thresholds (e.g., via the threshold determination module), and/or based on other processing related to the movement of the knee.

The feedback device may be coupled to the sleeve and may be communicably coupled to the processor. The feedback device may be configured to provide feedback to the wearer of the wearable device responsive to receiving a signal from the signal generation module. In some implementations, the feedback may be haptic feedback. In some implementations, the wearable device may comprise a plurality of feedback devices. In some of these implementations, a plurality of types of feedback may be provided. The types of feedback may comprise, for example, visual feedback, aural feedback, haptic feedback, and/or other types of feedback.

In some implementations, the wearable device may be configured for detecting and alerting a wearer to improper movement at a knee, an ankle, a wrist, an elbow, and/or other body joint. The wearable device may be used to detect sports injuries, train wearers how to properly move a body joint, help with rehabilitation of the body joint after an injury, and/or for other purposes related to tracking movement of the body joint. In some implementations where the wearable device is configured for a knee, the feedback device may be disposed adjacent to one of: a periosteum of the femoral condyle, a tibial plateau, or a fibula head of the knee of the wearer of the wearable device.

In some implementations, the wearable device may communicate with a mobile device of the user. The mobile device of the wearer may comprise an application configured to enable a physical processor of the mobile device to perform the functionality that the physical processor of the wearable device is configured to perform. The mobile device of the wearer may also generate and/or receive signals that may be generated and/or received by the wearable device. The mobile device of the wearer may also be configured to provide feedback to the wearer in lieu of (or in addition to) the feedback provided via the wearable device.

In some implementations, the wearable device may transmit sensory information sensed by the first sensor, baseline information obtained via the customization module, and/or other information obtained and/or processed by the wearable device to a central server. The central server may comprise a physical processor configured to execute computer-readable instructions to provide functionality. For example, the central server may be configured to aggregate sensory information from a plurality of wearable devices. Based on the aggregated sensory information, the central server may determine a plurality of initial thresholds. The central server may also send to the wearable device a recommendation of one or more revisions of thresholds for the wearer based on the information received from the wearable device. In some implementations, the central server may transmit signals to the wearable device to cause the wearable device to provide feedback to the wearer of the wearable device responsive to the central server detecting a potential improper movement. In some implementations, the central server may receive a request from a user interacting with the central server to send a signal to the wearable device.

These and other aspects, features, and characteristics of the present invention, as well as the functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

### BRIEF DESCRIPTION OF THE DRAWINGS

FIG. 1 illustrates an exemplary wearable device 100, according to an aspect of the invention.

FIG. 2 illustrates a schematic diagram of an inner surface of an exemplary wearable device 100, according to an aspect of the invention.

FIG. 3 illustrates a block diagram of an exemplary wearable device 100, according to an aspect of the invention.

FIG. 4A illustrates an exemplary wearable device 100a, according to an aspect of the invention.

FIG. 4B illustrates an exemplary wearable device 100b, according to an aspect of the invention.

FIG. 4C illustrates an exemplary wearable device 100c, according to an aspect of the invention.

FIG. 5 illustrates an exemplary method of detecting and alerting wearers to improper movements at a body joint, according to an aspect of the invention.

FIG. 6 illustrates an exemplary method of customizing thresholds for a wearer of the wearable device 100, according to an aspect of the invention.

FIG. 7 illustrates an exemplary method of revising thresholds for a wearer of the wearable device 100, according to an aspect of the invention.

### DETAILED DESCRIPTION

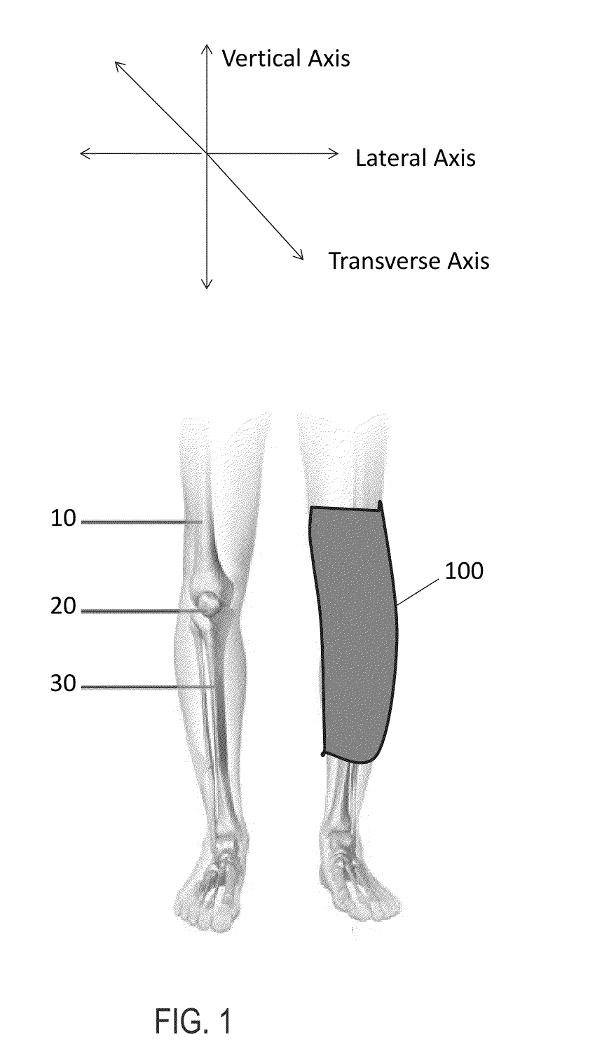

FIG. 1 illustrates an exemplary wearable device 100, according to an aspect of the invention. The wearable device 100 may be configured to detect and alert a wearer of the device 100 to a movement of the knee 20 that approaches and/or exceeds a threshold that is customized for the wearer. A threshold may comprise a combination of a bend angle and one or more lateral angle values at which the knee 20 makes or is about to make an improper movement. An improper movement comprises a movement that may result in injury, pain, and/or other undesirable consequence. A bend angle of a knee 20 may be an angle formed on the transverse axis by a first bone 30 (e.g., the tibia) relative to a second bone 10 (e.g., the femur). A lateral angle of the knee 20 may be an angle formed on the lateral axis by a horizontal displacement of the first bone 30 (e.g., the tibia) relative to the second bone 10 (e.g., the femur). A transverse axis may be perpendicular to a vertical axis and perpendicular to a horizontal axis. A knee 20 may move along the transverse axis by, for example, flexion and extension. A knee 20 may move along the vertical axis by, for example, elevation and depression. A knee 20 may move along the lateral axis by, for example, abduction and adduction.

For an individual bend angle, there may be more than one corresponding lateral angle at which the knee 20 may make an improper movement. For example, the individual angle may be associated with a corresponding inward lateral angle, at which the knee 20 makes an improper movement. The inward lateral angle may be an angle formed on the lateral axis by a horizontal inward displacement of the first bone 30 (e.g., the tibia) relative to the second bone 10 (e.g., the femur), where the first bone 30 has moved inward (e.g., towards the midline of the body) relative to the second bone 10. For example, the individual angle may be associated with a corresponding outward lateral angle, at which the knee 20 makes an improper movement. The outward lateral angle may be an angle formed on the lateral axis by a horizontal outward displacement of the first bone 30 (e.g., the tibia) relative to the second bone 10 (e.g., the femur), where the first bone 30 has moved outwards (e.g., away from the midline of the body) relative to the second bone 10. Accordingly, a threshold may comprise a bend angle value, an inward lateral angle value, and an outward lateral angle value.

In some examples, a threshold may comprise an individual bend angle associated with multiple inward lateral angles and multiple outward lateral angle values. For example, a combination of the individual bend angle and a first inward lateral angle of the plurality of inward lateral angles may be associated with an improper movement of the knee 20. A combination of the individual bend angle and one or more other inward lateral angles may be associated with a movement of the knee 20 in a direction in which an improper movement may occur. The threshold may also comprise, for an individual lateral angle, an indication whether the combination of the individual bend angle and the individual lateral angle is associated with an improper movement or with a potential occurrence of an improper movement. In some examples, the threshold may also comprise, for an individual lateral angle associated with the individual bend angle, feedback characteristics associated with feedback to provide to the wearer responsive to a determination that the threshold has been exceeded relative to the individual lateral angle. Feedback characteristics may comprise a number of types of feedback, one or more types of feedback, an intensity of feedback for individual ones of the one or more types of feedback, a length of time for which individual ones of the one or more types of feedback should be provided, and/or other characteristics related to providing feedback to the wearer of the device 100.

The wearable device 100 may determine a bend angle and a corresponding lateral angle of the knee 20. For example, the bend angle and corresponding lateral angle of the knee 20 may be determined by sensing the relative position of the first bone 30 and the second bone 10. The wearable device 100 may compare the determined bend angle and lateral angle with stored thresholds customized for the wearer. The thresholds may be stored on a non-transitory electronic storage media 130 coupled to the wearable device 100, may be stored on a mobile device of the wearer, may be stored at a remote data repository, and/or may be stored at another storage communicably coupled to the wearable device 100. The wearable device 100 may select a threshold from the plurality of thresholds with a bend angle that matches the determined bend angle. The wearable device 100 may then determine whether the determined lateral angle matches and/or exceeds one or more lateral angles associated with the bend angle of the threshold. The wearable device 100 may provide feedback based on the largest lateral angle associated with the bend angle of the threshold that the determined lateral angle matches and/or exceeds.

As mentioned above, the stored thresholds may be customized for a wearer of the wearable device 100. The wearable device 100 may be configured to obtain baseline information for the wearer's knee 20. The baseline information may be used to determine the stored threshold values. A threshold may be determined for an individual bend angle based on the value of the individual bend angle and the value of the corresponding lateral angle. The value of the corresponding lateral angle may be used, for example, as a median value to determine an inward lateral angle at which the knee 20 would be engaging in improper movement and an outward lateral angle at which the knee 20 would be engaging in improper movement. In some examples, a plurality of inward lateral angles and a plurality of outward lateral angles may be determined for the threshold as well.

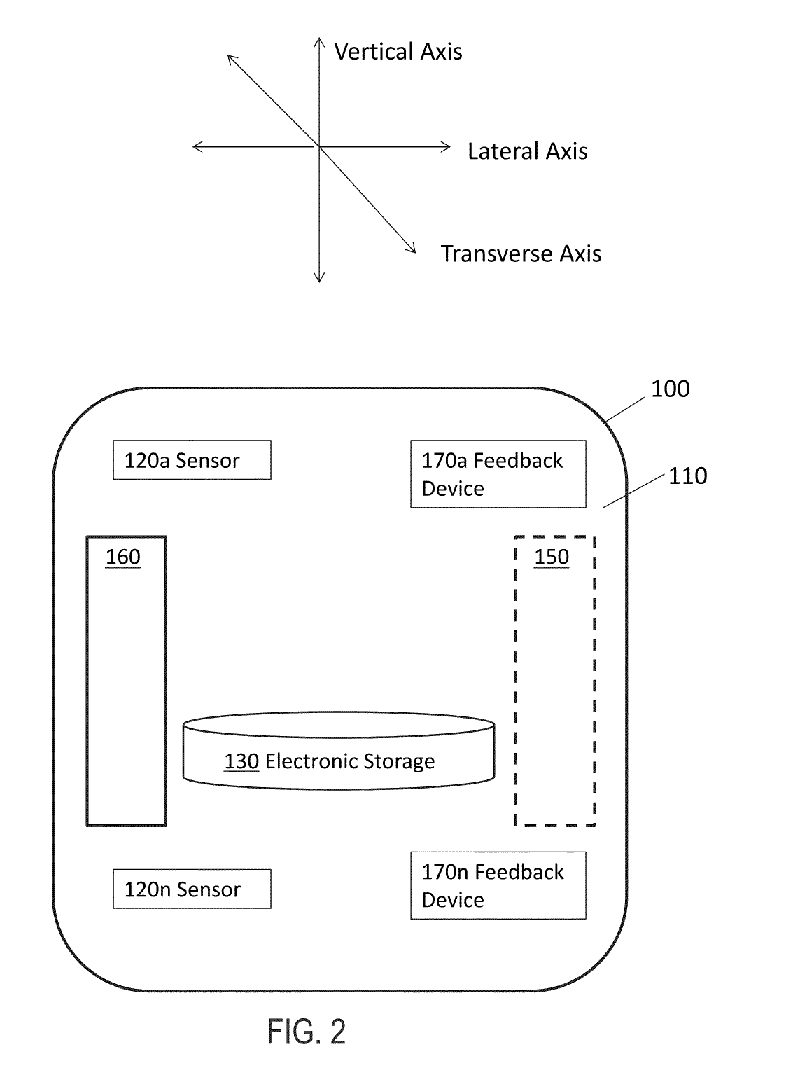

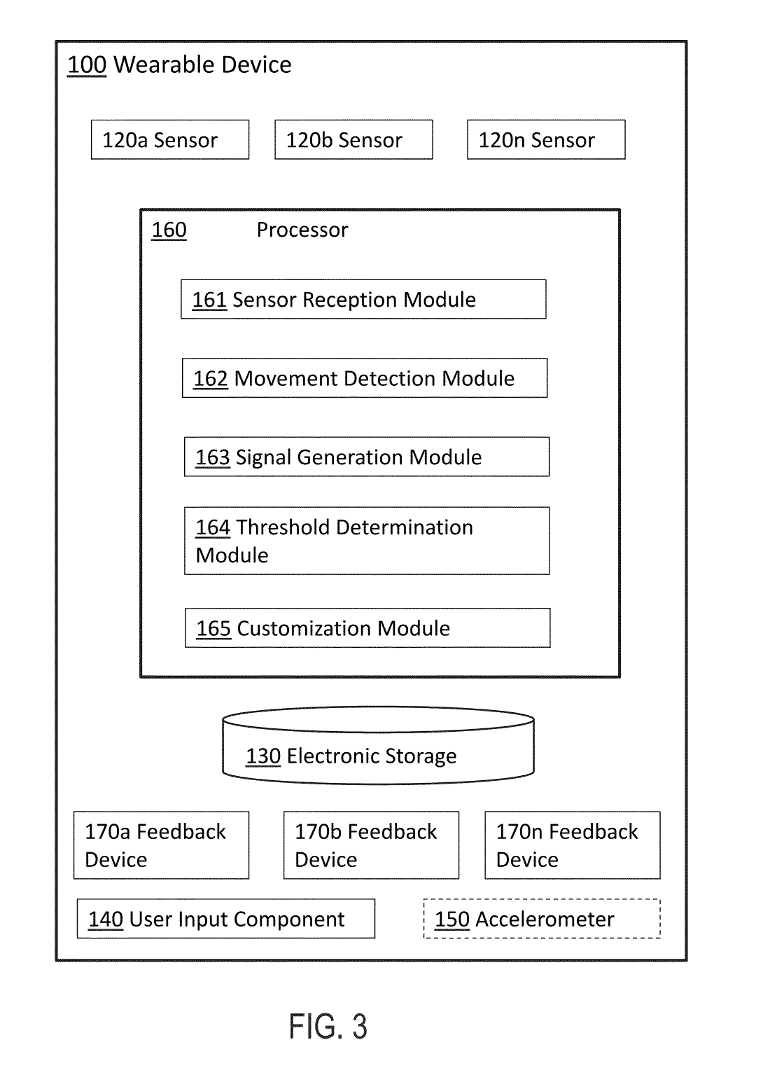

FIG. 2 illustrates a schematic diagram of an inner surface of an exemplary wearable device 100. FIG. 3 illustrates a block diagram of an exemplary wearable device 100.

As shown in FIGS. 2 and 3, a wearable device 100 may comprise, for example, a compliant sleeve 110, one or more sensors 120a, 120b, . . . , 120n, a non-transitory electronic storage media 130, a user input component 140, an accelerometer 150, a physical processor 160, one or more feedback devices 170a, 170b, . . . , 170n, and/or other components.

The compliant sleeve 110 may comprise a sleeve with an opening at a first end and an opening at a second end opposite to the first end, where the sleeve may be configured to encompass the knee 20 of a wearer of the wearable device 100. The sleeve may comprise a fabric sleeve. In some implementations, the physical processor 160 may be coupled with the fabric of the sleeve 110. For example, the physical processor and/or other chips may be integrated on a flexible printed circuit board that may be integrated into the fabric of the sleeve. The fabric may be lycra, a cotton blend, mesh, and/or other type of fabric that is compliant and with which the other components of the wearable device 100 may be coupled.

In some implementations, an enclosure coupled to the sleeve 110 may comprise the physical processor 160. The enclosure (or a separate enclosure) may comprise a power source for the wearable device 100. The power source may comprise, for example, a 5 volt power system with a rechargeable lithium ion battery, and/or other suitable power source for the wearable device 100. The enclosure may comprise PLA, ABS plastic, and/or other type of thermoplastic material. In some implementations, the enclosure may comprise a blue tooth communication system that may be communicably coupled to the physical processor 160, the one or more sensors 120a, 120b, . . . , 120n, the electronic storage media 130, the user input component 140, a mobile device of the wearer, and/or other components of the wearable device 100.

One or more sensors 120a, 120b, . . . , 120n may be coupled to the sleeve 110. The one or more sensors 120a, 120b, . . . , 120n may comprise, for example, bi-directional flex sensors, and/or other types of sensors that sense movement. In some implementations, four sensors may be placed at various positions of the wearable device 100 to sense movement of the knee 20. In some examples, sensory information sensed by the one or more sensors 120a, 120b, 120n may comprise information related to a relative position of the first bone 30 and the second bone 10. In some examples, the sensory information may comprise information related to a first bend angle and a first lateral angle formed by the first bone 30 and the second bone 10 at the first time. In some implementations, the sensory information may comprise information related to a first bend angle and a first lateral angle sensed at a first time and a second bend angle and a second lateral angle sensed at a second time after the first time. In some implementations, the sensory information may comprise a bend angle and corresponding lateral angle for each angle sensed during movement of the knee. In some implementations, the one or more sensors 120a, 120b, . . . , 120n may detect more than one type of movement. For example, the one or more sensors 120a, 120b, . . . , 120n may detect a bend angle and lateral angle during movement of the knee, may detect rotation of the body joint, rotation of the first bone 30 relative to the second bone 10, and/or may otherwise detect movement in multiple planes. In some implementations, a threshold may take into account a bend angle, corresponding lateral angle, and rotation of the first bone 30 relative to the second bone 10. The wearable device 100 may detect other types of multi-planar movement as well and is not limited to the examples described herein.

In some implementations, the one or more sensors 120a, 120b, . . . , 120n may also detect location of the wearable device 100. The one or more sensors 120a, 120b, . . . , 120n may comprise, for example, a GPS sensor, an RFID chip, an infrared sensor, a triangulation sensor, and/or other sensor that detects location. The sensory information may comprise a location at which the sensory information was obtained.

The one or more sensors 120a, 120b, 120n may be configured to transmit the sensory information related to the movement of the knee 20. The one or more sensors 120a, 120b, 120n may transmit the sensory information to the physical processor 160, to the electronic storage media 130 coupled to the wearable device 100, to a mobile device of the wearer, to a remote data repository, and/or to another storage communicably coupled to the wearable device 100.

The electronic storage media 130 may be configured to store thresholds for the wearer of the wearable device 100. In some implementations, the electronic storage media 130 may be configured to store the sensory information obtained via the wearable device 100. The electronic storage media 130 may be configured to store feedback information related to the provision of feedback as well. For example, the electronic storage media 130 may be configured may store sensory information that caused a signal to be generated for providing feedback, a location at which the feedback was provided, a date and/or time at which feedback was provided, feedback characteristics of the feedback provided, and/or other information related to the feedback provided.

The user input component 140 may be configured to receive input based on wearer interaction with the user input component 140. The user input component 140 may generate a signal based on the received input. The user input component 140 may be coupled to an outer surface of the wearable device 100. The signal generated by the user input component 140 may indicate one or more of: an indication to begin sensing the movement of the body joint, an indication to stop sensing the movement of the body joint, an indication to obtain baseline information from the wearer, and/or another indication to perform functionality of the wearable device.

The accelerometer 150 may be configured to detect acceleration of movement of the knee 20 and store the detected acceleration information. The acceleration information may comprise, for example, an acceleration value associated with the acceleration of the knee 20 at a first time. In some examples, the acceleration information may comprise a first acceleration value associated with acceleration of the knee 20 at a first time and a second acceleration value associated with acceleration of the knee 20 at a second time after the first time. In some implementations, the acceleration information may comprise a respective acceleration value associated with each bend angle and corresponding lateral angle in the sensory information.

The physical processor 160 may be coupled to the sleeve 110, communicably coupled to the one or more sensors 120a, 120b, . . . , 120n, communicably coupled to the electronic storage media 130, communicably coupled to the one or more feedback devices 170a, 170b, . . . , 170n, and/or communicably coupled to other components of the wearable device 100 and/or the mobile device of the wearer. The physical processor 160 may be configured to execute one or more program modules. The program modules may comprise, for example, a sensor reception module 161, a movement detection module 162, a signal generation module 163, a threshold determination module 164, a customization module 165, and/or other computer program modules.

The sensor reception module 161 may be configured to receive the sensory information. In some implementations, the sensor reception module 161 may determine a first bend angle and a first lateral angle based on the sensory information. In some implementations, the sensor reception module 161 may determine a plurality of bend angles and corresponding lateral angles based on the sensory information. For example, the sensor reception module 161 may determine a bend angle and corresponding lateral angle for each angle sensed during movement of the knee.

The movement detection module 162 may be configured to determine whether the first bend angle and the first lateral angle exceed a threshold for the wearer of the wearable device 100. The movement detection module 162 may select a threshold from the plurality of thresholds with a bend angle that matches the first bend angle. The movement detection module 162 may determine whether the first lateral angle is an inward lateral angle or an outward lateral angle. Responsive to a determination that the first lateral angle is an inward lateral angle, the movement detection module 162 may determine whether the first lateral angle matches and/or exceeds one or more inward lateral angles associated with the bend angle of the threshold. Responsive to a determination that the first lateral angle is an outward lateral angle, the movement detection module 162 may determine whether the first lateral angle matches and/or exceeds one or more outward lateral angles associated with the bend angle of the threshold.

In some implementations where the sensory information comprises a plurality of bend angles and corresponding lateral angles at a respective plurality of consecutive times, the movement detection module 162 may be configured to determine, for individual ones of the bend angles and corresponding lateral angles, whether the individual bend angle and corresponding lateral angle exceeds a corresponding threshold for the wearer of the wearable device 100.

In some implementations where the wearable device 100 comprises an accelerometer 150, the movement detection module 162 may be configured to determine whether the first lateral angle and the first bend angle exceed a corresponding threshold for the wearer of the wearable device 100 based on the acceleration of the knee 20 at a corresponding time.

The signal generation module 163 may be configured to generate a signal indicating that the knee 20 has made an improper movement, responsive to a determination by the movement detection module 162 that the first lateral angle matches and/or exceeds one or more lateral angles associated with the bend angle of the threshold. The signal generation module 163 may generate a signal based on the largest lateral angle associated with the bend angle of the threshold that the determined lateral angle matches and/or exceeds. The signal generation module 163 may transmit the generated signal to one or more feedback devices 170a, 170b, . . . , 170n coupled to the wearable device 100, to cause the one or more feedback devices 170a, 170b, . . . , 170n to provide feedback based on the generated signal. In some implementations, based on stored feedback characteristics associated with a largest lateral angle of the threshold which the first lateral angle matched and/or exceeded, the signal generation module 163 may generate one or more signals to cause the one or more feedback devices 170a, 170b, . . . , 170n to generate feedback according to the stored feedback characteristics.

The threshold determination module 164 may be configured to revise a threshold based on the bend angle and one or more lateral angles of the threshold and the stored sensory information. The threshold determination module 164 may determine that a second lateral angle associated with a second bend angle sensed at a second time did not match and/or exceed a corresponding second threshold. The threshold determination module 164 may determine that a third lateral angle associated with a third bend angle sensed at a third time immediately consecutive to the second time matched and/or exceeded a corresponding third threshold. The threshold determination module 164 may revise the second threshold for the second bend angle to include the second lateral angle with an indication that the combination of the second bend angle and the second lateral angle is associated with a potential occurrence of an improper movement.

In some implementations where the wearable device 100 comprises the accelerometer 150, the threshold determination module 164 may be configured to revise a threshold based on the bend angle and one or more lateral angles of the threshold, the stored sensory information, and acceleration information corresponding to the movement of the knee 20 at the bend angle. The threshold determination module 164 may determine that a second lateral angle associated with a second bend angle sensed at a second time did not match and/or exceed a corresponding second threshold. The threshold determination module 164 may determine that a third lateral angle associated with a third bend angle sensed at a third time immediately consecutive to the second time matched and/or exceeded a corresponding third threshold. The threshold determination module 164 may further determine, based on the acceleration information associated with the second bend angle, that the knee 20 would necessarily engage in movement in which it formed the third bend angle and the third lateral angle responsive to the knee 20 forming the second bend angle and the second lateral angle when the knee 20 moved at an acceleration included in the acceleration information. Responsive to that determination, the threshold determination module 164 may revise the second threshold for the second bend angle to include the second lateral angle with an indication that the combination of the second bend angle and the second lateral angle is associated with a potential occurrence of an improper movement.

The customization module 165 may be configured to obtain baseline information from the wearer responsive to a determination that a signal to obtain baseline information from the wearer was generated. The customization module 165 may be configured to determine thresholds for the wearer based on the baseline information.

In some implementations, the customization module 165 may be configured to determine a plurality of bend angles and corresponding lateral angles of the wearer's knees during a movement of the knee 20 from a straightened position to a bent position. The customization module 165 may provide an indication to the wearer of the wearable device 100 to move the knee 20 from a straightened position to a bent position. For example, the customization module 165 may send one or more signal to one or more feedback devices 170a, 170b, . . . , 170n to cause the one or more feedback devices 170a, 170b, . . . , 170n to provide the indication to the wearer of the wearable device 100.

The straightened position may comprise a position in which the tibia and the femur form a 180 degree angle relative to each other on the transverse axis. The bent position may comprise a position in which the tibia and the femur achieve a maximum bend along the transverse axis (and in which the bend angle of the knee along the transverse axis would be determined), a position in which the tibia and the femur comprise a 90 degree angle relative to each other on the transverse axis, and/or another position in which the tibia is bent with respective to the femur.

The customization module 165 may also send one or more signals to the one or more sensors 120a, 120b, . . . , 120n to sense the movement of the knee from the straightened to the bent position. The customization module 165 may receive sensory information from the one or more sensors 120a, 120b, . . . , 120n related to the movement of the knee from the straightened position to the bent position.

The customization module may be configured to determine baseline information from the received sensory information. The baseline information may comprise, for example, a bend angle and a lateral angle for each possible bend angle from the straightened position to the bent position, a bend angle and a lateral angle for a plurality of possible bend angles from the straightened position to the bent position, and/or other plurality of baseline measurements.

The customization module may determine one or more thresholds for the wearer of the wearable device 100 based on the baseline information. For example, a threshold may be determined for an individual bend angle based on the value of the individual bend angle and the value of the corresponding lateral angle. The value of the corresponding lateral angle may be used, for example, as a median value to determine an inward lateral angle at which the knee would be engaging in improper movement and an outward lateral angle at which the knee would be engaging in improper movement. In some examples, a plurality of inward lateral angles and a plurality of outward lateral angles may be determined for the threshold as well.

In some implementations, the wearable device 100 may be unable to provide feedback to the wearer based on the movement of the wearer's knee 20 until the wearable device 100 obtains baseline information for the wearer. In some implementations, the electronic storage media 130 of the wearable device 100 may store a plurality of initial thresholds. The wearable device 100 may revise, replace, and/or otherwise change the plurality of initial thresholds based on receiving baseline information (e.g., via the customization module 165), revising the thresholds (e.g., via the threshold determination module 164), and/or based on other processing related to the movement of the knee 20.

The one or more feedback devices 170a, 170b, . . . , 170n may be coupled to the sleeve 110 and may be communicably coupled to the physical processor 160. The one or more feedback devices 170a, 170b, . . . , 170n may comprise one or more types of feedback devices. For example, the one or more feedback devices 170a, 170b, . . . , 170n may comprise haptic feedback devices, visual feedback devices, aural feedback devices, odor-emitting devices, and/or other types of feedback devices. The one or more feedback devices 170a, 170b, . . . , 170n may be configured to provide feedback to the wearer of the wearable device 100 responsive to receiving a signal from the physical processor 160. In some implementations, the feedback may be haptic feedback. In some of these implementations, a plurality of types of feedback may be provided. The types of feedback may comprise, for example, visual feedback, aural feedback, haptic feedback, scent feedback, and/or other types of feedback. In some implementations, a pattern of feedback, including one or more types of feedback, may be provided. In some implementations, one or more of the feedback devices 170a, 170b, . . . , 170n may be disposed adjacent to one of: a periosteum of the femoral condyle, a tibial plateau, or a fibula head of the knee 20 of the wearer of the wearable device 100.

In some implementations, the wearable device 100 may communicate with a mobile device of the user. The mobile device of the wearer may comprise an application configured to enable a physical processor of the mobile device to perform the functionality that the physical processor of the wearable device 100 is configured to perform. The mobile device of the wearer may also be configured to generate and/or receive signals that may be generated and/or received by the wearable device 100. The mobile device of the wearer may also be configured to provide feedback to the wearer in lieu of (or in addition to) the feedback provided via the wearable device 100.

In some implementations, the wearable device 100 may transmit sensory information sensed by the one or more sensors 120a, 120b, . . . , 120n, baseline information obtained via the customization module 165 of the physical processor 160, feedback information related to feedback provided to the wearer of the wearable device 100, and/or other information obtained and/or processed by the wearable device 100 to a central server. The central server may comprise a physical processor configured to execute computer-readable instructions to provide functionality. For example, the central server may be configured to aggregate sensory information from a plurality of wearable devices 100. Based on the aggregated sensory information, the central server may determine a plurality of initial thresholds. The central server may also send to the wearable device 100 a recommendation of one or more revisions of thresholds for the wearer based on the information received from the wearable device 100. In some implementations, the mobile device of the wearer may be configured to provide the functionality provided by the central server.

In some implementations, the central server may transmit signals to the wearable device 100 to cause the wearable device to provide feedback to the wearer of the wearable device 100 when the central server detects a potential improper movement. In some implementations, the central server may receive a request from a user interacting with the central server to send a signal to the wearable device 100.

The central server may also determine whether improper movement (or potential improper movement) occurs at or near specific locations for a particular wearer of the wearable device 100 or for more than one wearer of the wearable device 100. For example, the central server may determine whether improper movement (or potential improper movement) occurs based on the aggregated sensory information, feedback information related to feedback provided to the wearer of the wearable device 100, and/or other information related to the wearable device 100.

In some implementations, the central server may provide a notification to one or more wearers of the wearable device 100 responsive to determining that the one or more wearers engage in improper or potentially improper movement near a particular location. In some implementations, the central server may provide a notification to one or more entities responsible for the upkeep of the property comprising the location. The central server may also correlate a location with a repeated improper movement for a wearer of the wearable device 100. In some implementations, based on the correlation, the central server may augment the stored thresholds with location information. The wearable device 100 may be caused to provide feedback to the wearer when the wearer approaches the location. For example, the wearable device 100 may be caused to provide the feedback regardless of the sensed movement of the wearable device 100.

Processor(s) 160 is configured to provide processing capabilities in wearable device 100. As such, processor 160 may include one or more of a digital processor, an analog processor, a digital circuit designed to process information, an analog circuit designed to process information, a state machine, and/or other mechanisms for electronically processing information. In some implementations, processor 160 may be a custom processor designed for the wearable device 100. For example, processor 160 may comprise an Arduino 32 bit 16 mhz processor with 4 analog channel and 6 digital channels.

Although processor 160 is shown in FIG. 1 as a single entity, this is for illustrative purposes only. In some implementations, processor 160 may include a plurality of processing units. These processing units may be physically located within the same device, or processor 160 may represent processing functionality of a plurality of devices operating in coordination. The processor 160 may be configured to execute modules 161, 162, 163, 164, and 165. Processor 160 may be configured to execute modules 161, 162, 163, 164, and 165 by software; hardware; firmware; some combination of software, hardware, and/or firmware; and/or other mechanisms for configuring processing capabilities on processor 160. As used herein, the term “module” may refer to any component or set of components that perform the functionality attributed to the module. This may include one or more physical processors during execution of processor readable instructions, the processor readable instructions, circuitry, hardware, storage media, or any other components.

It should be appreciated that although modules 161, 162, 163, 164, and 165 are illustrated in FIG. 3 as being implemented within a single processing unit, in implementations in which processor includes multiple processing units, one or more of modules 161, 162, 163, 164, and 165 may be implemented remotely from the other modules. The description of the functionality provided by the different modules 161, 162, 163, 164, and 165 described below is for illustrative purposes, and is not intended to be limiting, as any of modules 161, 162, 163, 164, and 165 may provide more or less functionality than is described. For example, one or more of modules 18161, 162, 163, 164, and 165 may be eliminated, and some or all of its functionality may be provided by other ones of modules 161, 162, 163, 164, and 165. As another example, processor 160 may be configured to execute one or more additional modules that may perform some or all of the functionality attributed below to one of modules 161, 162, 163, 164, and 165.

Non-transitory electronic storage media 130 may comprise non-transitory storage media that electronically stores information. The electronic storage media 130 may include one or both of system storage that is provided integrally (i.e., substantially non-removable) with wearable device 100 and/or removable storage that is removably connectable to wearable device 100 via, for example, a port (e.g., a USB port, a firewire port, etc.) or a drive (e.g., a disk drive, etc.). Electronic storage media 130 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage media 130 may include one or more virtual storage resources (e.g., cloud storage, a virtual private network, and/or other virtual storage resources). Electronic storage media 130 may store software algorithms, information determined by processor 160, information received from wearable device 100, information received from a mobile device of the wearer, and/or other information that enables wearable device 100 to function as described herein.

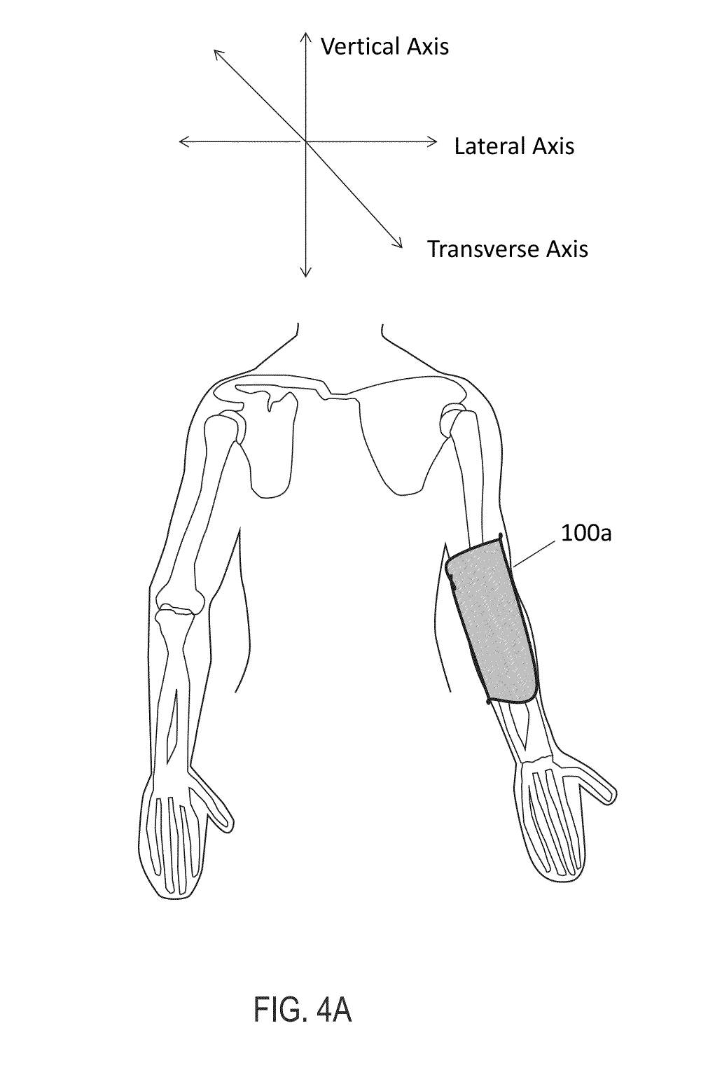

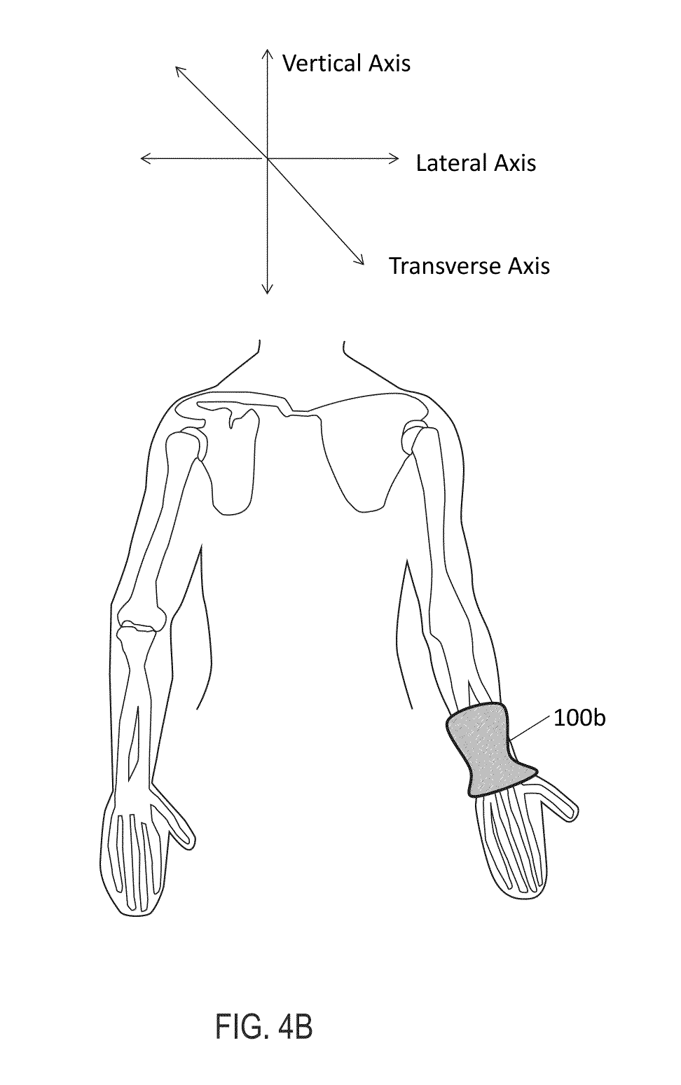

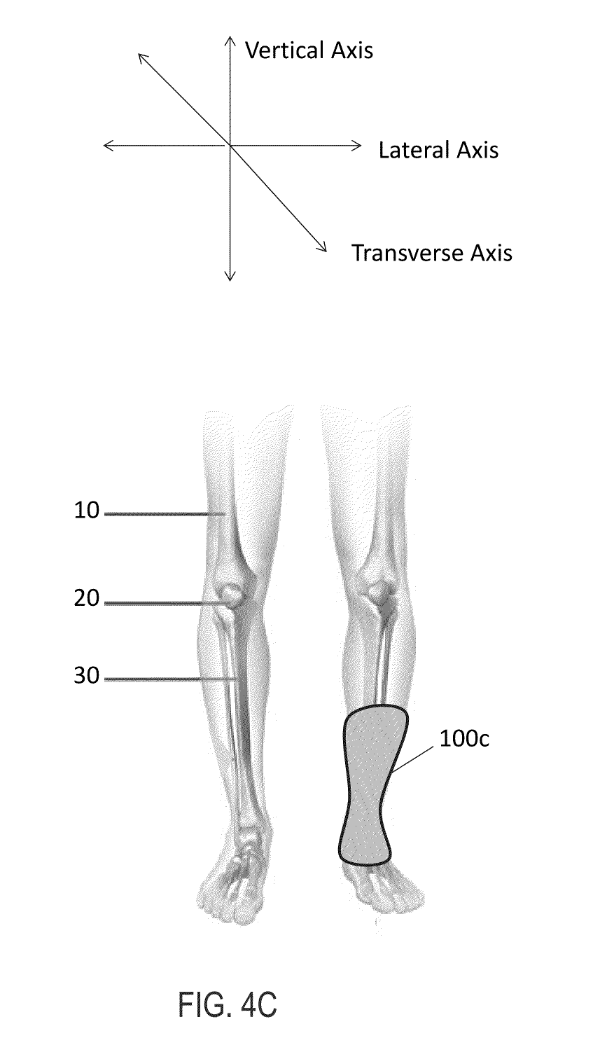

In some implementations, the wearable device 100 may be configured for an ankle, a wrist, an elbow, and/or other body joint. For example, as shown in FIG. 4A, a wearable device 100a may be configured to detect and alert a wearer of the device 100a to a movement of an elbow that approaches or exceeds a threshold that is customized for the wearer. In another example, as shown in FIG. 4B, a wearable device 100b may be configured to detect and alert a wearer of the device 100b to a movement of a wrist that approaches or exceeds a threshold that is customized for the wearer. In yet another example, as shown in FIG. 4C, a wearable device 100b may be configured to detect and alert a wearer of the device 100c to a movement of an ankle that approaches or exceeds a threshold that is customized for the wearer.

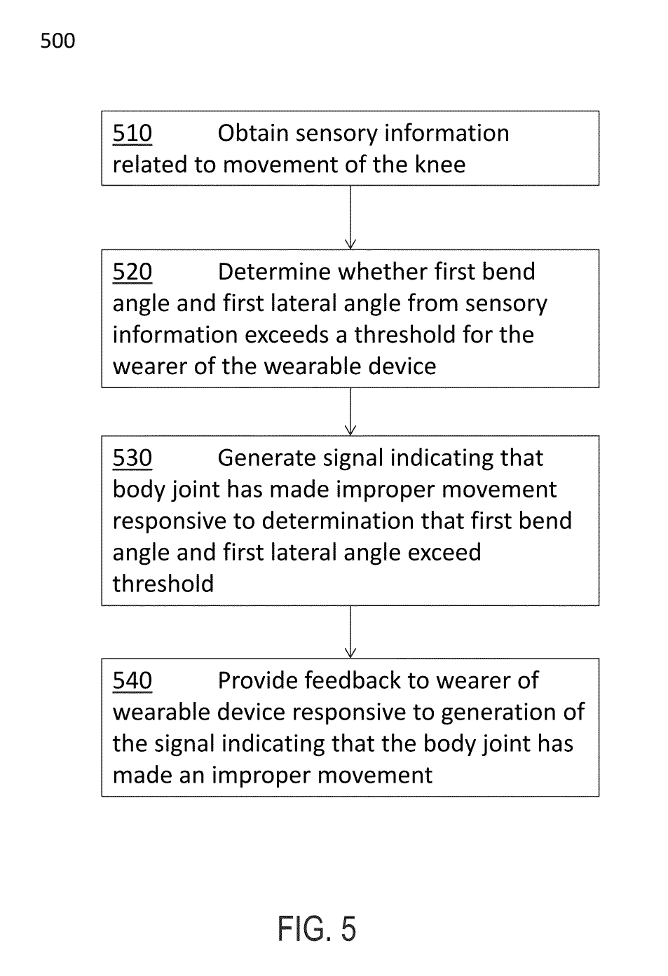

FIG. 5 illustrates an exemplary method 500 of detecting and alerting wearers to improper movements at the knee 20 of the wearer of the wearable device 100. The described operations of FIG. 5 and other figures may be accomplished using some or all of the components of the wearable device 100 described in detail above and, in some implementations, various operations may be performed in different sequences. In other implementations, additional operations may be performed along with some or all of the operations shown in FIG. 5 and the other figures. In yet other implementations, one or more operations may be performed simultaneously. In yet other implementations, one or more combinations of various operations may be performed. Some implementations may not perform all of the operations described with relation to FIG. 5 and other figures. Accordingly, the operations described are exemplary in nature and, as such, should not be viewed as limiting.

In some embodiments, the described operations of FIG. 5 and other figures may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the described operations of FIG. 5 and other figures in response to instructions stored electronically on an electronic storage medium. The one or more processing devices may include one or more devices configured through hardware, firmware, and/or software to be specifically designed for execution of one or more of the described operations of FIG. 5 and other figures.

At an operation 510, sensory information related to the movement of the knee may be obtained. In some implementations, operation 510 may be performed by one or more sensors the same as or similar to the one or more sensors 120a, 120b, . . . , 120n of the wearable device 100 (shown in FIG. 3 and described above).

At an operation 520, a determination may be made as to whether a first bend angle and a first lateral angle from the sensory information exceed a corresponding threshold for the wearer of the wearable device 100. In some implementations, operation 520 may be performed by a movement detection module the same as or similar to movement detection module 162 of the physical processor 160 of the wearable device 100 (shown in FIG. 3 and described above).

At an operation 530, a signal may be generated that indicates that the knee has made an improper movement (or is about to make an improper movement). The signal may be generated responsive to a determination that the first bend angle and the first lateral angle exceed a corresponding threshold. In some implementations, operation 530 may be performed by a signal generation module the same as or similar to signal generation module 163 of the physical processor 160 of the wearable device 100 (shown in FIG. 3 and described above).

At an operation 540, feedback may be provided to the wearer of the wearable device 100 responsive to the generation of the signal at operation 530. In some implementations, operation 540 may be performed by one or more feedback devices the same as or similar to the one or more feedback devices 170a, 170b, . . . , 170n of the wearable device 100 (shown in FIG. 3 and described above).

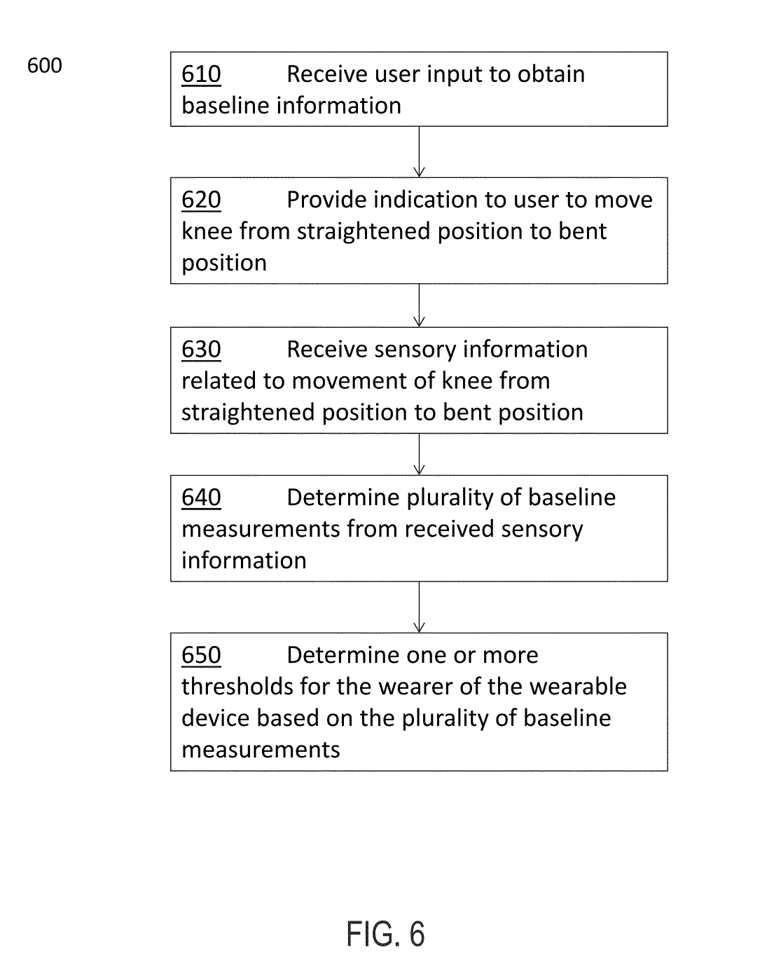

FIG. 6 illustrates an exemplary method of customizing thresholds for a wearer of the wearable device 100.

At an operation 610, user input may be obtained that indicates a desire to obtain baseline information for the wearer. In some implementations, operation 610 may be performed by a user input component and/or a signal generation module the same as or similar to the user input component 140 and/or a signal generation module 163 of the physical processor 160 of the wearable device 100 (shown in FIG. 3 and described above).

At an operation 620, an indication may be provided to a wearer of the wearable device 100 to move the knee from a straightened position to a bent position. In some implementations, operation 620 may be performed by a customization module the same as or similar to customization module 165 of the physical processor 160 of the wearable device 100 (shown in FIG. 3 and described above).

At an operation 630, sensory information related to the movement of the knee from the straightened position to the bent position may be received. In some implementations, operation 630 may be performed by a customization module the same as or similar to customization module 165 of the physical processor 160 of the wearable device 100 (shown in FIG. 3 and described above).

At an operation 640, baseline information (including a plurality of baseline measurements) may be determined from the received sensory information. In some implementations, operation 640 may be performed by a customization module the same as or similar to customization module 165 of the physical processor 160 of the wearable device 100 (shown in FIG. 3 and described above).

At an operation 650, one or more thresholds may be determined for the wearer of the wearable device 100 based on the plurality of baseline measurements in the baseline information. In some implementations, operation 640 may be performed by a customization module the same as or similar to customization module 165 of the physical processor 160 of the wearable device 100 (shown in FIG. 3 and described above).

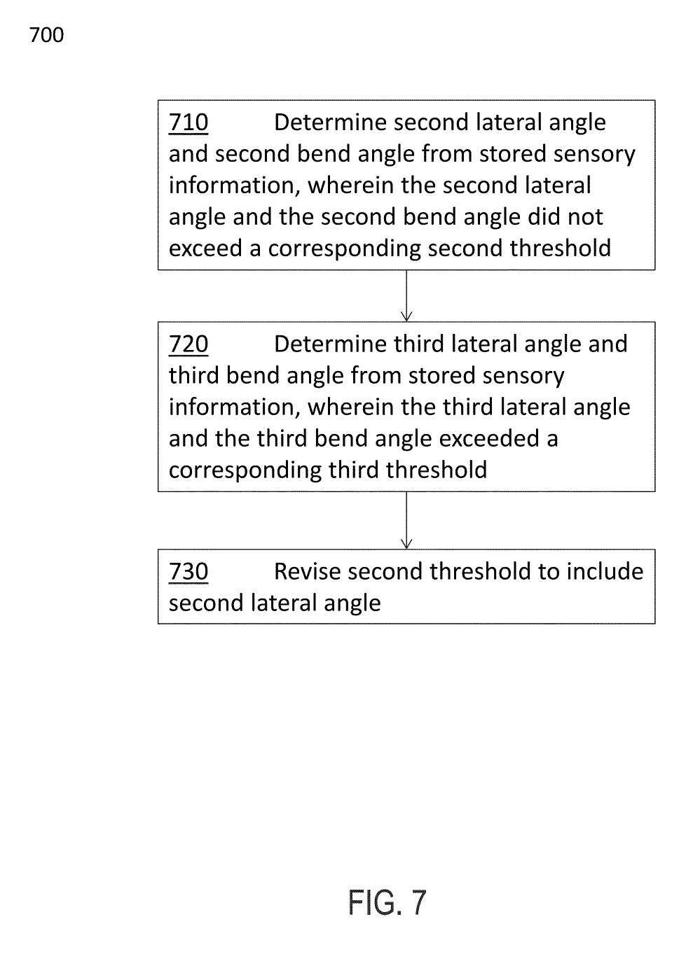

FIG. 7 illustrates an exemplary method of revising thresholds for a wearer of the wearable device 100.

At an operation 710, a second bend angle and second lateral angle may be determined from stored sensory information. The second bend angle and second lateral angle may be determined to have not exceeded a corresponding second threshold. In some implementations, operation 710 may be performed by a threshold determination module the same as or similar to threshold determination module 164 of the physical processor 160 of the wearable device 100 (shown in FIG. 3 and described above).

At an operation 720, a third bend angle and third lateral angle may be determined from stored sensory information. The third bend angle and third lateral angle may be determined to have matched and/or exceeded a corresponding second third. In some implementations, operation 720 may be performed by a threshold determination module the same as or similar to threshold determination module 164 of the physical processor 160 of the wearable device 100 (shown in FIG. 3 and described above).

At an operation 730, the second threshold may be revised to include the second lateral angle. The threshold may comprise an indication with the second lateral angle that the combination of the second bend angle and the second lateral angle is associated with a potential occurrence of an improper movement. In some implementations, operation 730 may be performed by a threshold determination module the same as or similar to threshold determination module 164 of the physical processor 160 of the wearable device 100 (shown in FIG. 3 and described above).

Aspects and implementations described herein as including a particular feature, structure, or characteristic, but every aspect or implementation may not necessarily include the particular feature, structure, or characteristic. Further, when a particular feature, structure, or characteristic is described in connection with an aspect or implementation, it will be understood that such feature, structure, or characteristic may be included in connection with other aspects or implementations, whether or not explicitly described. Thus, various changes and modifications may be made to the provided description without departing from the scope or spirit of the invention. As such, the specification and drawings should be regarded as exemplary only, and the scope of the invention to be determined solely by the appended claims.

What is claimed is:

1. A wearable device for a body joint, the wearable device comprising:
a compliant sleeve configured to encompass the body joint of a wearer of the wearable device;
a first sensor coupled to the sleeve, the first sensor configured to:
obtain sensory information related to the movement of the body joint, the sensory information comprising information related to a first bend angle formed along a transverse axis by a first bone connected to the body joint relative to a second bone connected to the body and a first lateral angle formed by the body joint along a lateral axis by a horizontal displacement of the first bone relative to the second bone at a first time, wherein the transverse axis is perpendicular to the lateral axis and perpendicular to a vertical axis;
transmit the sensory information;
a physical processor coupled to the sleeve and communicably coupled to the first sensor, the physical processor configured to execute one or more program modules, the program modules comprising:
a sensor reception module configured to receive the sensory information;
a movement detection module configured to determine whether the first bend angle and the first lateral angle exceed a threshold for the wearer of the wearable device; and
a signal generation module configured to generate a signal indicating that the body joint has made an improper movement responsive to a determination by the movement detection module that the first bend angle and the first lateral angle exceed a threshold for the wearer of the wearable device; and
a feedback device coupled to the sleeve and communicably coupled to the processor, the feedback device configured to provide feedback to the wearer of the wearable device responsive to generation of the signal by the signal generation module.

2. The wearable device of claim 1, further comprising:
a non-transitory electronic storage media configured to store a plurality of thresholds, wherein an individual threshold comprises an individual bend angle, one or more inward lateral angles, and one or more outward lateral angles,
wherein an individual inward lateral angle comprises an angle formed on the lateral axis by a horizontal displacement of the first bone relative to the second bone in a direction towards a midline of a body of the wearer and wherein an individual outward lateral angle comprises an angle formed on the lateral axis by a horizontal displacement of the first bone relative to the second bone in a direction away from the midline of the body of the wearer.

3. The wearable device of claim 2, wherein the electronic storage media is coupled to the wearable device and communicably coupled to the processor.

4. The wearable device of claim 2, wherein the movement detection module is configured to determine whether the first bend angle and the first lateral angle exceed a threshold for the wearer of the wearable device by:
selecting the threshold from the plurality of thresholds, where the threshold comprises a bend angle that matches the first bend angle;
determine whether the first lateral angle exceeds one of: the one or more inward lateral angles or the one or more outward lateral angles.

5. The wearable device of claim 2, further comprising:
a user input component coupled to an outer surface of the wearable device, the user input component configured to:
receive input based on wearer interaction with the user input component; and
generate a signal based on the received input, wherein the signal may indicate one or more of: an indication to begin sensing the movement of the body joint, an indication to stop sensing the movement of the body joint, or an indication to obtain baseline information from the wearer.

6. The wearable device of claim 2, wherein the computer program modules comprise:
a customization module configured to obtain baseline information from the wearer responsive to a determination that the signal to obtain baseline information from the wearer was generated, wherein the customization module is configured to obtain baseline information from the wearer by:
providing an indication to the wearer to begin moving the body joint from a straightened position to a bent position;
receiving sensory information from the sensor related to movement of the body joint from the straightened position to the bent position;
determining a plurality of baseline measurements from the received sensory information; and
determining one or more thresholds for the wearer based on the plurality of baseline measurements.

7. The wearable device of claim 2, wherein the electronic storage media is configured to store the sensory information received via the sensor reception module, wherein the computer program modules comprise:
a threshold determination module configured to revise the individual threshold based on the stored sensory information.

8. The wearable device of claim 7, wherein the threshold determination module is configured to revise the individual threshold by:
determining, from the stored sensory information, a second lateral angle and a second bend angle sensed at a second time, wherein the second lateral angle and the second bend angle did not exceed a corresponding second threshold;
determining, from the stored sensory information, a third lateral angle and a third bend angle sensed at a third time after the second time, wherein the third lateral angle and the third bend angle exceeded a corresponding third threshold.

9. The wearable device of claim 1, wherein the sensory information comprises a plurality of bend angles and corresponding lateral angles sensed at a respective plurality of consecutive times, and
wherein the movement detection module is configured to determine, for individual ones of the bend angles and corresponding lateral angles, whether the individual bend angle and corresponding lateral angle exceed a corresponding threshold for the wearer of the wearable device.

10. The wearable device of claim 1, wherein the body joint is a knee, and wherein the feedback device is disposed adjacent to one of: a periosteum of the femoral condyle, a tibial plateau, or a fibula head of the knee of the wearer of the wearable device.

11. The wearable device of claim 1, wherein the sleeve comprises a fabric sleeve, and wherein the processor is integrated with the fabric of the sleeve.

12. The wearable device of claim 1, further comprising:
an accelerometer configured to detect acceleration of movement of the body joint and store acceleration information related to acceleration of movement of the body joint, the acceleration information comprising a first acceleration value detected by the accelerometer at the first time.

13. The wearable device of claim 12, wherein the movement detection module is configured to determine whether the first lateral angle and the first bend angle exceed the threshold for the wearer of the wearable device based on the acceleration value of the body joint at the first time.

14. The wearable device of claim 13, wherein the electronic storage media is configured to store the sensory information and the acceleration information detected by the accelerometer, and
wherein the computer program modules comprise:
a threshold determination module configured to revise the threshold based on the stored acceleration information.

15. The wearable device of claim 14, wherein the threshold determination module is configured to revise the threshold by:
determining, from the stored sensory information, a second lateral angle and a second bend angle sensed at a second time, wherein the second lateral angle and the second bend angle did not exceed a corresponding second threshold;
determining, from the stored sensory information, a third lateral angle and a third bend angle sensed at a third time after the second time, wherein the third lateral angle and the third bend angle exceeded a corresponding third threshold;
determining, based on second acceleration information detected at the second time, whether the knee would move from forming the second lateral angle and second bend angle to forming the third bend angle and third lateral angle;
responsive to a determination that the knee would move from forming the second lateral angle and second bend angle to forming the third bend angle and third lateral angle, revising the second threshold to include the second lateral angle.

16. A wearable device for a body joint, the wearable device comprising:
a compliant sleeve configured to encompass the body joint of a wearer of the wearable device;
a non-transitory electronic storage media configured to store a plurality of thresholds, wherein an individual threshold comprises an individual bend angle, one or more inward lateral angles, and one or more outward lateral angles, wherein an individual bend angle comprises an angle formed along a transverse axis by a first bone connected to the body joint relative to a second bone connected to the body, wherein an individual inward lateral angle comprises an angle formed on a lateral axis by a horizontal displacement of the first bone relative to the second bone in a direction towards a midline of a body of the wearer, wherein an individual outward lateral angle comprises an angle formed on the lateral axis by a horizontal displacement of the first bone relative to the second bone in a direction away from the midline of the body of the wearer, and wherein the transverse axis is perpendicular to the lateral axis and perpendicular to a vertical axis;
a user input component coupled to an outer surface of the wearable device, the user input component configured to:
receive input based on wearer interaction with the user input component; and
generate a signal based on the received input, wherein the signal may indicate one or more of: an indication to begin sensing the movement of the body joint, an indication to stop sensing the movement of the body joint, or an indication to obtain baseline information from the wearer; and
a physical processor coupled to the sleeve, the physical processor configured to execute one or more program modules, the program modules comprising:
a customization module configured to obtain baseline information from the wearer responsive to a determination that the signal to obtain baseline information from the wearer was generated, wherein the customization module is configured to obtain baseline information from the wearer by:
providing an indication to the wearer to begin moving the body joint from a straightened position to a bent position;
receiving sensory information from the sensor related to movement of the body joint from the straightened position to the bent position;
determining a plurality of baseline measurements from the received sensory information; and
determining one or more thresholds for the wearer based on the plurality of baseline measurements.

17. A method of determining improper movement of a body joint using a wearable device, the method comprising:
obtaining, by a first sensor coupled to the wearable device, sensory information related to the movement of the body joint of a wearer of the wearable device, wherein the wearable device is configured to encompass the body joint of the wearer, the sensory information comprising information related to a first bend angle formed along a transverse axis by a first bone connected to the body joint relative to a second bone connected to the body and a first lateral angle formed by the body joint along a lateral axis by a horizontal displacement of the first bone relative to the second bone at the first time, wherein the transverse axis is perpendicular to the lateral axis and perpendicular to a vertical axis;
determining, by a physical processor coupled to the wearable device and coupled to the first sensor, whether the first bend angle and the first lateral angle exceed a threshold for the wearer of the wearable device; and
generating, by the physical processor, a signal indicating that the body joint has made an improper movement responsive to a determination that the first bend angle and the first lateral angle exceed a threshold for the wearer of the wearable device; and
providing, via a feedback device coupled to the wearable device and communicably coupled to the physical processor, feedback to the wearer of the wearable device responsive to generation of the signal.

18. The method of claim 17, further comprising:
storing, at a non-transitory electronic storage media, a plurality of thresholds, wherein an individual threshold comprises an individual bend angle, one or more inward lateral angles, and one or more outward lateral angles,
wherein an individual inward lateral angle comprises an angle formed on the lateral axis by a horizontal displacement of the first bone relative to the second bone in a direction towards a midline of a body of the wearer and wherein an individual outward lateral angle comprises an angle formed on the lateral axis by a horizontal displacement of the first bone relative to the second bone in a direction away from the midline of the body of the wearer.

19. The method of claim 18, further comprising:
receiving input based on wearer interaction with a user input component coupled to the wearable device;
generating a signal based on the received input, wherein the signal may indicate one or more of: an indication to begin sensing the movement of the body joint, an indication to stop sensing the movement of the body joint, or an indication to obtain baseline information from the wearer;
obtaining baseline information from the wearer responsive to a determination that the signal to obtain baseline information from the wearer was generated, wherein obtaining baseline information comprises:
providing an indication to the wearer to begin moving the body joint from a straightened position to a bent position;
receiving sensory information from the sensor related to movement of the body joint from the straightened position to the bent position;
determining a plurality of baseline measurements from the received sensory information; and
determining one or more thresholds for the wearer based on the plurality of baseline measurements.

20. The method of claim 18, further comprises:
storing, at the electronic storage media, the obtained sensory information;
revising a threshold of the plurality of thresholds based on the stored sensory information by:
determining, from the stored sensory information, a second lateral angle and a second bend angle sensed at a second time, wherein the second lateral angle and the second bend angle did not exceed a corresponding second threshold;
determining, from the stored sensory information, a third lateral angle and a third bend angle sensed at a third time after the second time, wherein the third lateral angle and the third bend angle exceeded a corresponding third threshold; and
revising the second threshold to include the second lateral angle.
